SEQUENCE LISTING (3 sequences)

SEQ ID NO: 1 - DNA, 10 bp, Artificial Sequence
  Synthetic Linker
aggcgcgcct

SEQ ID NO: 2 - DNA, 21 bp, Artificial Sequence
  Primer
cctctacaaatgtgtgatggc

SEQ ID NO: 3 - DNA, 23 bp, Artificial Sequence
  Primer
gcgcaccatcttcttcaaggacg

(19) United States
(12) Patent Application Publication
(10) Pub. No.: US 2002/0178460 A1
(43) Pub. Date: Nov. 28, 2002

(54) Transgenic mice expressing fluorescent protein

(75) Inventors: Grigori N. Enikolopov, Cold Spring Harbor, NY (US); John Mignone, Bronxville, NY (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Cold Spring Harbor Laboratory

(21) Appl. No.: 10/150,509

(22) Filed: May 16, 2002

Related U.S. Application Data
(63) Continuation of application No. PCT/US00/31150, filed on Nov. 14, 2000.
(63) Continuation-in-part of application No. 09/444,335, filed on Nov. 19, 1999.

(51) Int. Cl. A01K 67/027

(52) U.S. Cl. 800/18

(57) ABSTRACT

Non-human transgenic mammals are produced which have, incorporated in their genome, DNA which includes a regulatory sequence of a mammalian nestin gene, operably linked to a gene coding for a marker/reporter protein. The regulatory sequence can include a promoter and a sequence present in the second intron of the mammalian nestin gene. Preferably, the marker/reporter protein is a fluorescent protein, for example a green fluorescent protein, modified for enhanced fluorescence. Multipotent and, in particular, neural stem and progenitor cell populations are observed in the organs of the non-transgenic mammal or progeny thereof. Multipotent stem and progenitor cells are isolated directly from the non-human transgenic mammal, progeny or embryo thereof, for example by FACS, without culture passages.

### RELATED APPLICATIONS

This application is a continuation of International Application No. PCT/US00/31150, which designated the United States and was filed on Nov. 14, 2000, published in English, which is a Continuation-in-Part of U.S. patent application Ser. No. 09/444,335, filed on Nov. 19, 1999. The entire teachings of the above applications are incorporated herein by reference.

### BACKGROUND OF THE INVENTION

It is generally believed that cells of the central nervous system (CNS) originate from the neuroectoderm in the neural plate on the dorsal side of the embryo. After the neural tube closure, cells of the neuroepithelium differentiate to form neuronal and glial cells. One characteristic of neural stem and progenitor cells is the expression of nestin, an intermediate filament protein.

Neural stem and progenitor cells are generally obtained from the developing or adult brain and cultivated in vitro as cell cultures, typically for long periods of time. Colonies that express certain markers, for example, nestin, can be identified, isolated and expanded. However, cells obtained by this procedure undergo repeated passages in culture and are no longer the originally harvested cells. Properties characterizing the original cells may be lost. For example, prolonged in vitro cultivation may result in progenitor cells which, though not yet fully differentiated into neural cells (neurons, astrocytes, oligodentrocytes, etc.) have lost the true multipotent neural stem cell character. Furthermore, the technique outlined above does not allow isolation of multipotent early progenitor cells that retain regional specificity and express markers specific for one or another region of the central nervous system, while at the same time preserving their capacity to generate differentiated cells of varied type.

Therefore, a need exists for methods to isolate stem and progenitor cells from an animal or embryo directly, without the need of prolonged in vitro cultivation.

### SUMMARY OF THE INVENTION

The invention relates to a non-human transgenic mammal, progeny or embryo thereof which has integrated into its genome DNA comprising a regulatory sequence of a mammalian nestin gene. The nestin gene is expressed in proliferating multipotent stem and progenitor cells and is downregulated once the multipotent stem and progenitor cells differentiate and loose their multipotent character. Operably linked to the regulatory sequence of the mammalian nestin gene is a gene coding for a fluoresecent protein. In one embodiment, the multipotent stem and progenitor cells are neural stem and progenitor cells. In another embodiment, the fluorescent protein is selectively expressed in multipotent stem and progenitor cells. In yet another embodiment of the invention, the DNA includes a promoter, a gene coding for a fluorescent protein (e.g., green fluorescent protein) and a second intron sequence of a mammalian nestin gene, wherein the gene coding for the fluorescent protein is expressed in multipotent stem and progenitor cells of the non-human transgenic mammal, progeny or embryo thereof.

The invention further relates to a method for producing a non-human transgenic mammal expressing a fluorescent protein in multipotent stem and progenitor cells. The method comprises introducing into a fertilized egg of a non-human mammal, DNA comprising a regulatory sequence of a mammalian nestin gene operably linked to a gene coding for a fluorescent protein that is expressed in multipotent stem and progenitor cells of the non-human mammal. The fertilized egg having the DNA comprising a regulatory sequence of a mammalian nestin gene operably linked to a gene coding for a fluorescent protein is introduced into a non-human mammal of the same species which is allowed to produce progeny. The progeny are non-human transgenic mammals. The method further includes selecting from the non-human transgenic mammal progeny, obtained as described above, non-human mammal progeny whose multipotent stem and progenitor cells express the fluorescent gene.

In addition, the invention relates to an expression construct or vector and also to a cell comprising it. The expression construct includes a promoter sequence, a gene coding for green fluorescent protein and a regulatory sequence present in the second intron of a mammalian nestin gene. In a preferred embodiment, the promoter is a promoter of the nestin gene. The invention also is related to cells which include such an expression construct or vector.

The invention further relates to a method for measuring a multipotent stem and progenitor cell population in an animal organ or region thereof. The method comprises measuring cells which fluoresce from the organ or region thereof of a non-human transgenic mammal which has integrated into its genome DNA comprising a regulatory sequence of a mammalian nestin gene operably linked to a gene coding for a fluorescent protein, wherein the gene coding for the fluorescent protein is expressed in multipotent stem and progenitor cells of the non-human transgenic mammal. Cells which fluoresce are multipotent stem and progenitor cells.

Also related to the invention is a method for obtaining primary, non-cultured, multipotent stem and progenitor cells comprising isolating cells which express a marker/reporter protein (e.g., a fluorescent protein) from a non-human transgenic mammal, progeny or embryo thereof, which has integrated into its genome DNA comprising a regulatory sequence of a mammalian nestin gene operably linked to a gene coding for the marker/reporter protein (e.g., a fluorescent protein). The gene coding for the marker/reporter protein is expressed in multipotent stem and progenitor cells of the non-human transgenic mammal, progeny or embryo thereof. In one embodiment, the multipotent stem and progenitor cells are neural stem and progenitor cells. In another embodiment, the marker/reporter protein is selectively expressed in multipotent stem and progenitor cells. In yet another embodiment, the marker/reporter protein is a fluorescent protein and fluorescent cells are isolated by fluorescent activated cell sorting. The invention also related to cells obtained or isolated by these methods.

Moreover, the invention relates to a method for assessing a compound's ability to promote multipotent stem and progenitor cell differentiation. The method comprises contacting live multipotent stem and progenitor cells capable of differentiation, which have integrated into their genome DNA comprising a regulatory sequence of a mammalian nestin gene operably linked to a gene coding for a marker/reporter protein (e.g. fluorescent protein) wherein the gene coding for the marker/reporter protein is expressed in multipotent stem and progenitor cells, with a compound to be assessed; determining a marker/reporter protein measurement (e.g. fluorescence) of the live cells in the presence of the compound; and comparing the marker/reporter protein measurement of cells in the presence of the compound to marker/reporter protein measurement of live control cells. A decrease or absence of marker/reporter measurement of live cells in the presence of the compound compared to the the marker/reporter measurement of live control cells is indicative of the compound's ability to promote multipotent stem and progenitor cell differentiation.

Another method of the present invention is a method for assessing a compound's toxicity to multipotent stem and progenitor cells. A compound's toxicity can be assessed by its ability to kill stem and progenitor cells. The method comprises contacting live stem and progenitor cells, which have integrated into their genome DNA comprising a regulatory sequence of a mammalian nestin gene operably linked to a gene coding for a marker/reporter protein (e.g., fluorescent protein), wherein the gene coding for the marker/reporter protein is expressed in stem and progenitor cells, with a compound to be assessed. A marker/reporter protein measurement of live fluorescent cells in the presence of the compound is measured and compared to the marker/reporter protein measurement of control cells. A decrease or absence of the marker/reporter protein measurement of live cells in the presence of the compound, compared to the measurement of control cells, is indicative of the compound's toxicity to multipotent stem and progenitor cells.

Other aspects of the invention are directed to assessing a compound for its ability to promote differentiation of live totipotent stem and progenitor cells into multipotent stem and progenitor cells. In the method, live totipotent stem and progenitor cells which have integrated into their genome DNA comprising a regulatory sequence of a mammalian nestin gene, operably linked to a gene coding for a marker/reporter protein (e.g. a gene coding for a fluorescent protein), wherein the marker/reporter gene is expressed in the cells, is contacted with the compound to be assessed. A measurement of the marker/reporter protein (e.g., fluorescence) of the cells in the presence of the compound is determined and compared to the measurement of the marker/reporter protein in control cells. An increase in measured marker/reporter protein of cells in the presence of the compound compared to the measurement of the marker/reporter protein of control cells is indicative of the differentiation of the totipotent cells into multipotent stem and progenitor cells.

Similarly, the present invention relates to a method of assessing a compound's ability to promote differentiation of multipotent stem and progenitor cells into neural cells. In this method, if the cells under investigation are multipotent stem and progenitor cells, a decrease in a measurement of a marker/reporter protein (e.g., fluorescence) of cells in the presence of the compound compared to the the measurement of the marker/reporter protein of control cells is indicative of the compound's ability to promote differentiation of the multipotent stem and progenitor cells into neural cells.

The invention has numerous advantages. For example, by practicing the invention, intact multipotent stem and progenitor cells as well as intact neural stem and progenitor cells are obtained directly, without prolonged in vitro cultivation. The cells produced retain regional specificity while preserving their capacity to generate differentiated cells of varied types. Furthermore, the cells produced by the method of the invention can be used to assess compounds that promote differentiation and compounds that are toxic towards the cells. In addition, the method of the invention allows studying multipotent stem and progenitor cells in animal models. For instance, neural stem and progenitor cells can be monitored in vivo in order to follow the effects of compounds administered in vivo, to investigate neurogenesis in the normal brain during both embryonic and post embryonic stages, after brain injury or after transplantation experiments. Cell migration during normal organ development and after transplantation can also be detected. In addition, the invention provides methods for assessing the ability of compounds to promote differentiation of stem and progenitor cells and of neural stem and progenitor cells. Since intact cells can be isolated from specific organs or regions thereof and since the regulatory elements directing gene expression integrated into the genome of the cells is known, cell-specific genes, proteins, surface antigens, and other cell-specific markers, potentially unique to cell subsets, can be identified.

### BRIEF DESCRIPTION OF THE DRAWINGS

FIGS. 1A-1C are a diagram showing the preparation of an expression construct of one embodiment of the invention.

FIG. 2 illustrates an expression construct including a nestin promoter, a gene sequence coding for green fluorescence protein an a second intron sequence of the nestin gene.

FIGS. 3A-3C and 3G show the fluorescent activated cell sorting (FACS) results from control cells.

FIGS. 3D-3F and 3H-3N show FACS of cells obtained from a non-human transgenic mammal.

### DETAILED DESCRIPTION OF THE INVENTION

The invention is related to a non-human transgenic mammal or progeny thereof which has integrated into its genome DNA comprising a regulatory sequence of a mammalian nestin gene. Operably linked to the regulatory sequence of the mammalian nestin gene is a gene coding for a marker/reporter protein, such as, for example, a fluorescent protein. The marker/reporter protein is expressed in multipotent stem and progenitor cells of the non-human transgenic mammal, progeny or embryo thereof.

Any suitable non-human mammal can be used to produce the non-human transgenic mammals described herein. As used herein, the term “non-human transgenic mammal” includes the newly born, young offsprings, developing adults, embryos of the non-human transgenic mammal, as well as newly born, young offsprings, developing adults or embryos of a progeny of the non-human transgenic mammal. Examples of non-human transgenic mammals and their progeny include mouse, rat, dog, monkey, as well as any other suitable non-human mammalian species. A preferred mammal is mouse.

Generally, a stem cell is thought of as a cell with the capacity to divide asymmetrically, producing one copy of itself and one, more committed daughter cell. Often, stem cells are thought of as undifferentiated cells with the ability to proliferate, to exhibit self-maintenance, to generate a large number of progeny and to generate new cells in response to injury or disease. Generally, a progenitor cell is a more committed cell which divides symmetrically and can be differentiated into more mature morphotypes.

As used herein, the phrase “multipotent stem and progenitor cells” are cells which express nestin. Generally, multipotent stem and progenitor cells have regional specificity and are capable, upon differentiation, of generating cell types characteristic of a certain organ or tissue present in a mammalian organism. Neural stem and progenitor cells are one example of multipotent stem and progenitor cells. Upon, differentiation, neural stem and progenitor cells give rise to neural cells, such as glial cells and neurons.

Embryonic or totipotent precursors of multipotent stem and progenitor cells are referred to herein as “totipotent stem and progenitor cells”. Due to their totipotent character, these cells are capable of differentiating into cells characteristic of any organ or tissue in the mammalian organism. As used herein, totipotent stem and progenitor cells are precursors of multipotent cells, do not possess regional specificity and can be distinguished from multipotent stem and progenitor cells by the fact that they do not express nestin.

Nestin is an intermediate filament protein; in particular, it defines a distinct sixth class of intermediate filament protein. Nestin is expressed, for example, in neural stem and progenitor cells. Its expression diminishes as neural stem and progenitor cells differentiate into neural cell types. In healthy mammals, fully differentiated cells of the CNS, such as neurons, astrocytes and oligodentrocytes, do not generally express nestin. However, nestin expression has been identified in some CNS tumors and after injury to the adult spinal cord or optic nerve. In the case of injury, nestin production has been observed in reactive astrocytes and in cells close to the central canal in the spinal cord. It has been reported (C. B. Johansson et al. Cell, 96:25-34 (1999)) that, in adult mammals, cavity lining cells, such as ependymal cells, express nestin, in particular following spinal cord injury.

Nestin expression also has been observed in multipotent stem and progenitor cells other than neural stem and progenitor cells. As reported, for example, by Kobayashi, M., et al., Pediatr. Res. 43(3): 386-392 (1998), nestin is expressed in muscle precursors; however, mature muscle cells do not express nestin (Zimmerman, L., et al., Neuron (US), 12(1): 11-24 (1994). Nestin expression has also been linked to developing organs such as, for example, the liver (Niki, T., et al., Hepatology 29(2): 520-527 (1999)), tooth (Terling, C., et al., Int. J. Dev Biol 39(6): 947-956 (1995), and heart (Kachinsky, A. M., et al., J. Histochem Cytochem 43(8): 843-847 (1995). In addition, nestin expression can occur in multipotent stem and progenitor cells of the pancreas, intestinal tract, and retina.

A variety of nestin genes or sequences thereof can be used in the compositions and methods of the present invention. Examples of suitable mammalian nestin genes include rat nestin gene, human nestin gene, mouse nestin gene and nestin genes specific to any other mammalian species. In a preferred embodiment of the invention, the mammalian nestin gene is rat nestin gene.

Nestin genes of mammalian origin have been isolated and sequenced. For example, nucleotide sequences of rat and human nestin genes and deduced amino acid sequences of the corresponding nestin proteins are disclosed in U.S. Pat. No. 5,338,839 issued on Aug. 16, 1994 to McKay, et al., which is incorporated herein by reference in its entirety. Regulatory elements of the nestin gene, e.g., rat, are discussed, for example, in Zimmerman, L. et al., Neuron, 12: 11-24 (1994), which is incorporated herein by reference in its entirety.

As used herein, “a regulatory sequence of a mammalian nestin gene” includes one or more regulatory sequences of the nestin gene which, when operably linked to a gene encoding a protein, expresses the protein in multipotent stem and progenitor cells. In one embodiment of the invention, the non-human transgenic mammal or progeny thereof has integrated into its genome DNA including a regulatory sequence of a mammalian nestin gene wherein the regulatory sequence is such that the marker/reporter protein is expressed in multipotent stem and progenitor cells. In another embodiment of the invention, the regulatory sequence is such that the marker/reporter protein is selectively expressed in multipotent stem and progenitor cells (e.g., the central nervous system). In yet another embodiment, the regulatory sequence selectively expresses in neural stem and progenitor cells.

In a preferred embodiment, the regulatory sequence includes the entire second intron sequence of the mammalian nestin gene. Shorter sequences of the second intron also can be employed. Examples of suitable shorter sequences which can be employed are known in the art. For example, in European Journal of Neuroscience, 9: 452-462 (1997), hereby incorporated by reference in its entirety, Lothian and Lendahl, showed that transgenic mice generated with the most conserved 714 bp in the 3′ portion of the second human intron or with the complete, 1852 bp, human intron gave very similar nestin-like expression pattern and concluded that the important control elements reside in the 714 bp element. In Experimental Cell Research (United States), 248 (2): 509-519 (1999), hereby incorporated by reference in its entirety, Lothian, et al. showed that a 374-bp region in the second intron of the human nestin gene is sufficient and a 120-bp sequence in this region is required for the expression of the nestin gene in neural cells of the embryonic CNS.

Optionally, the regulatory sequence can further include elements present in the first intron of the mammalian nestin gene. The entire sequence of the first intron or shorter sequences thereof can be employed. As discussed by Zimmerman, et al. in Neuron, 12: 11-24 (1994), hereby incorporated by reference in its entirety, independent and cell-type specific elements in the first and second introns of the nestin gene direct reporter gene expression to the developing muscle and neural precursors, respectively.

The regulatory sequence of a mammalian nestin gene, as defined herein, can include any suitable promoter. In one embodiment, the promoter can be a nestin promoter. In a preferred embodiment, the nestin promoter is obtained from the same mammalian nestin gene as the regulatory sequence. Suitable promoters also include promoter sequences which are functional in mammalian cells, yeast, bacteria and insect cells. Examples of suitable promoter include but are not limited to, polyhedrin, 3-phosphoglycerate kinase, metallothionein, retroviral LTR, SV40 and TK promoters and others known in the art.

In the compositions and methods of the present invention, the regulatory sequence of a mammalian nestin gene, as defined above, is operably linked to a gene coding for a marker/reporter protein. The gene coding for the marker/reporter protein is expressed in multipotent stem and progenitor cells of the non-human transgenic mammal, progeny or embryo thereof. In one embodiment of the invention, the marker/reporter protein is selectively expressed in multipotent stem and progenitor cells. As used herein, the term “selectively expressed” means that the marker/reporter protein is expressed to a detectable level predominantly in multipotent stem and progenitor cells. In another embodiment, the marker/reporter protein is expressed to a detectable level in neural stem and progenitor cells. In yet another embodiment, the marker/reporter protein is selectively expressed in neural stem and progenitor cells.

Marker/reporter proteins for use in the composition and methods of the present invention are known to those of skill in the art. Marker/reporter proteins for which there are convenient and simple assay methods are preferred. Examples include, but are not limited to, luminescent proteins, fluorescent proteins, enzymes, cell surface proteins and other proteins known in the art.

A preferred marker/reporter protein which can be employed is a fluorescent protein. Examples of suitable fluorescent proteins include, but are not limited to green fluorescent protein (GFP), modified or enhanced green fluorescent protein (EGFP), yellow fluorescent protein, cyan FP, blue FP, red FP and their enhanced versions (Clontech) and any other luminscent or fluorescent protein that can emit light. In a preferred embodiment, the marker/reporter protein is a fluorescent protein such as green fluorescent protein (GFP). In another, the GFP is modified for enhanced fluorescence. GFP as well as mutants of GFP are known to those skilled in the art. For example, proteins exhibiting green fluorescence are described in U.S. Pat. No. 5,491,084 and in U.S. Pat. No. 5,804,387, which are incorporated herein by reference in their entirety. In still another embodiment of the invention, the fluorescent protein modified for enhanced fluorescence is EGFP (enhanced green fluorescent protein) which can be obtained from the pEGFP-N1 plasmid supplied by Clontech. Briefly, the plasmid included 190 silent base changes from human codon preferences; there was a conversion of the ATG codon for better Kozak consensus and amino acid substitutions: Phe64-Leu and Ser65-Thr.

The invention is also related to a method for producing a non-human transgenic mammal which expresses a fluorescent protein in multipotent stem and progenitor cells, comprising introducing into a fertilized egg of a non-human mammal, DNA comprising a regulatory sequence of a mammalian nestin gene, as defined above, operably linked to a gene coding for a fluorescent protein, such as described above, that is expressed in multipotent stem and progenitor cells of the non-human mammal. The fertilized egg is introduced into a non-human mammal, preferably of the same species, which is allowed to produce progeny which are non-human transgenic mammal progeny. The method also comprises selecting from the non-human transgenic mammal progeny those progeny whose multipotent stem and progenitor cells express the fluorescent gene. In one embodiment of the invention, the method comprises selecting from the non-human transgenic mammal progeny those progeny whose neural stem and progenitor cells express the fluorescent gene. Genes expressing GFP or GFP modified for enhanced fluorescence, e.g., EGFP, are preferred.

Another aspect of the invention is related to a non-human transgenic mammal which expresses a fluorescent protein in stem and progenitor cells produced by a method comprising introducing into a fertilized egg of a non-human mammal, DNA comprising a regulatory sequence of a mammalian nestin gene, as defined above, operably linked to a gene coding for a fluorescent protein, such as described above, that is expressed in stem and progenitor cells of the non-human mammal; by introducing the fertilized egg into a non-human mammal, preferably of the same species, which is allowed to produce progeny which are non-human transgenic mammal progeny and by selecting from the non-human transgenic mammal progeny those progeny whose stem and progenitor cells express the fluorescent gene. In one embodiment of the invention, the non-human transgenic mammal progeny selected are those progeny whose neural stem and progenitor cells express the fluorescent gene.

In a preferred embodiment, the DNA comprising a regulatory sequence of a mammalian nestin gene operably linked to a fluorescent protein is an expression construct or vector which comprises a promoter sequence, preferably a promoter sequence of a mammalian nestin gene, a gene coding for green fluorescent protein and a regulatory sequence present in the second intron of a nestin gene. An example of such a construct, methods for producing as well as methods for introducing the construct into the fertilized egg of the non-human mammal are further described below.

A cell or cells which comprise(s) the expression construct of the invention can be isolated and can be further employed. For instance, such cells can be studied and characterized in vitro or can be used in experiments designed to monitor cellular development and/or differentiation. Cells which comprise the construct of the invention also can be transplanted into organs of recipient animals. The cells of the invention can be employed in other experimental methods known in the art.

The invention is also related to assessing the presence of multipotent stem and progenitor cells in the organism, organs or a region thereof of the non-human transgenic mammal, in its progeny or in the non-human transgenic embryo of the invention. In a preferred embodiment, the non-human transgenic mammal employed has integrated into its genome DNA comprising a regulatory sequence of the mammalian nestin gene opreably linked to a gene coding for a fluorescent protein. Populations of multipotent stem and progenitor cells can be assessed by viewing or measuring fluorescence from an organ or region thereof the non-human transgenic mammal, progeny or embryo thereof. The presence of fluorescent cells also can be assessed in organs subjected to trauma, during tissue or organ regeneration, during various treatments, before and after transplantation, and during various stages of development in the presence or absence of various environmental factors or stimuli. In vivo effects of compounds administered to animal models and affecting multipotent stem and progenitor cells can be evaluated by using the non-human transgenic mammal of the invention and by measuring the fluorescence of an organ or region thereof and comparing it to the fluorescence of the organ or region thereof in control animals.

Another aspect of the invention is also related to a method for obtaining or isolating primary, non-cultured multipotent (e.g., neural) stem and progenitor cells. Such cells are also referred to herein as intact, fresh, or simply primary multipotent stem and progenitor cells. Such cells are obtained from a non-human transgenic mammal of the invention, from a progeny thereof or from a non-human transgenic mammalian embryo, directly, without culture passages. The use of these terms, however, is not intended to preclude the possibility of in vitro studies of such fresh, intact or primary cells. Accordingly, once obtained from the non-human transgenic mammal or progeny thereof, the primary multipotent stem and progenitor cells isolated according to the method of the invention can be further cultivated in vitro using techniques known by those skilled in the art.

A method of obtaining live primary multipotent stem and progenitor cells comprises isolating cells which express the marker/reporter protein defined above from a non-human transgenic mammal, progeny or embryo thereof which has integrated into its genome DNA comprising a regulatory sequence of a mammalian nestin gene operably linked to a gene coding for a marker/reporter protein wherein the gene coding for the marker/reporter protein is expressed in multipotent stem and progenitor cells of the non-human transgenic mammal, progeny or embryo thereof. Another method of obtaining live primary multipotent stem and progenitor cells comprises isolating fluorescent cells from a non-human transgenic mammal, progeny or embryo thereof which has integrated into its genome DNA comprising a regulatory sequence of a mammalian nestin gene operably linked to a gene coding for a fluorescent protein wherein the gene coding for the fluorescent protein is expressed in multipotent stem and progenitor cells of the non-human transgenic mammal, progeny or embryo thereof.

Multipotent stem and progenitor cells present in organs or regions thereof can be isolated by the compositions and methods of the invention. In a preferred embodiment, the isolated cells are neural stem and progenitor cells. Multipotent stem and progenitor cells present in other organs and expressing nestin, for example muscle precursor cells, can also be purified (e.g., highly enriched).

In a preferred embodiment, cells expressing a fluorescent protein can be isolated using fluorescent activated cell sorting (FACS). With proteins modified for enhanced fluorescence, the brightness of the transgene-expressing cells is very high and FACS proves to be a quick and efficient procedure. FACS techniques are known to those skilled in the art. The use of FACS for sorting cells is discussed, for example, in U.S. Pat. No. 5,804,387. In a preferred embodiment, the fluorescent protein is green fluorescent protein enhanced for fluorescence and identified as EGFP. Primary, non-cultured EGFP expressing cells can be isolated from the intact organism by FACS in less than an hour, typically in 10 to 30 minutes.

Other methods can be employed in obtaining or isolating cells which have integrated in their genome DNA comprising a regulatory sequence of a mammalian nestin gene operably linked to a marker/reporter protein can be employed. Examples include, but are not limited to using the fluorescent (Herzberg) β-gal substrate as described by Nolan, G. P., et al., Proc. Natl. Acad. Sci. USA 85(8): 2603-2607 (1988) or the method described by Stemple, D. L., et al., Cell, 71(6): 973-85 (1992).

Methods relying on the expression of a cell surface marker specific to the nestin protein could also be employed. In one embodiment of the invention, a cell surface marker, such as, for example, a receptor, can be employed as the marker/reporter protein instead of the GFP. Multipotent stem and progenitor cells can then be purified by employing fluorescence or tagged antibody techniques, such as FACS or magnetic beads bound to antibodies in conjunction with magnetic separation. Petri dishes with antibodies fixed to the surface can also be used to preferentially adhere the multipotent stem and progenitor cells to the surface of the petri dish.

Once isolated, nestin-expressing cells can be further studied and characterized by techniques known to those skilled in the art. In one embodiment of the invention, RNA and proteins are separated from isolated primary cells. Proteins specific to the isolated cells can be identified, for example, by two dimensional electrophoresis or by isoelectrofocusing.

In another embodiment of the invention, genes characterizing the intact cells isolated as described above are identified as well. For example, this can be accomplished by versions of gene chip technology. Examples of gene chip approaches known to those skilled in the art include the Affimetrix or Synteni approaches. One procedure for identifying such genes includes preparing a catalog or library of, for example, genes, cDNA, expressed sequence tags (EST) in the isolated cells and comparing the catalog against genes expressed in non-fluorescent cells. The non-fluorescent cells may be cells that are in an earlier stage of development, (e.g., totipotent cells) than nestin-expressing cells or may be cells that have differentiated beyond the nestin-expressing stage. Similarly, the catalog can be compared to genes expressed by non-fluorescent cells in specific organs or regions thereof.

In still another embodiment of the invention, surface antigens specific to the cells isolated as described above are identified. Techniques for identifying surface cell specific surface antigens are known to those skilled in the art. These techniques include, for example, immunizing animals with the isolated cells and obtaining antibodies directed against cell specific antigens from the immunized animals.

In yet another embodiment of the invention, cells isolated according to the invention are transplanted into animals. In particular, the isolated cells can be transplanted in specific organs or regions thereof. Techniques for accomplishing the transplantation of isolated cells into an animal are known to those skilled in the art. The animal may be of the same species as the non-human transgenic mammal of the invention. Alternatively, the animal can be of a different species. Examples of animals include mouse, rat, monkey and many others.

The non-human transgenic mammal or progeny thereof described above and cells isolated according to the invention can be employed to identify compounds that affect the differentiation of totipotent and multipotent stem and progenitor cells. As used herein, the term compound includes, for example, pharmaceutical formulations such as drugs and other biologically active compounds that may be administered in the treatment, diagnosis or prophylaxis of various medical indications or conditions. Such compounds are generally referred to herein as “therapeutic agents”. Preferred therapeutic agents include growth factors and neutrophins. Other compounds which can be employed include but are not limited to: small molecules (such as organic or organometallic molecules), vitamins, proteins, peptides, polypeptides, viruses, nucleic acids, hormones (such as growth factors), enzymes (for example, nitric oxide synthase), and other biological compounds of natural or recombinant DNA origin which may be implicated in cellular development or differentiation.

As described herein, the present invention further relates to methods of identifying whether a compound (i) promotes multipotent stem and progenitor cell differentiation; (ii) is toxic to multipotent stem and progenitor cells; (iii) promotes differentiation of totipotent to multipotent stem and progenitor cells; and (iv) promotes differentiation of multipotent stem and progenitor cells into neural cells. The methods include detecting or measuring the expression of a marker/reporter protein. Methods of detecting or measuring marker/reporter gene expression are known to those of skill in the art. Luminescence, fluorescence, enzymatic activity (e.g. β-gal), magnetic beads and other methods of antibody-based purification, fluorescent activated cell sorting, differential centrifugation and other assay methods, known in the art can be employed. A preferred marker/reporter gene is one which expresses a fluorescent protein, as described above. Fluorescence is measured by techniques and equipment known to those skilled in the art. Excitation and emission wavelengths are selected in accordance to the fluorescent marker/reporter protein used and are known in the art. In one embodiment, GFP (excitation wavelength of about 395 nm and emission wavelength of about 509 nm) is employed. In another embodiment, EGFP (excitation wavelength of about 488 nm and an emission wavelength is about 507 nm) is employed.

Compounds screened or evaluated can be administered or delivered in vivo to the non-human transgenic mammal of the invention. The compounds can also be studied in vitro. As used herein, the phrases “contacting live multipotent stem and progenitor cells”, “contacting live totipotent stem and progenitor cells” and “contacting live neural stem and progenior cells” with a compound includes in vitro treatment of cells as well as in vivo administration of the compound.

A marker/reporter protein measurement of organs or regions thereof in animals who have received the compound in vivo can be compared to the marker/reporter protein measurement of the corresponding organs or regions thereof in control animals that have not received the compound. Another suitable method of evaluating the effects of compounds administered in vivo includes harvesting and isolating cells from a sacrificed non-human transgenic mammal who had received the compound and comparing the marker/reporter protein measurement in the isolated cells to control cells obtained from non-human transgenic mammals who have not received the compound. Compounds administered in vivo and their effects on the cells can be evaluated, for example, by observing tissue fluorescence changes or by FACS of cells harvested from the sacrificed non-human transgenic mammals.

Compounds can also be screened in vitro by employing cells isolated from the non-human transgenic mammal or cells (e.g., totipotent stem and progenitor cells; multipotent stems and progenitor cells) transfected with a construct comprising a promoter sequence, a gene encoding a marker/reporter protein and a regulatory sequence present in the second intron of a mammalian nestin gene by the methods described above. The cells can be contacted with a compound to be assessed and the marker/reporter protein (e.g. fluorescent protein) of the cells in the presence of the compound is measured and compared to the marker/reporter protein measured in control cells. As known by those skilled in the art, the sample of cells in the presence of the compound is matched to the control cell sample in such a manner that any difference in the marker/reporter protein measurement (e.g., fluorescence) can be attributed solely to the effect of the compound.

In one embodiment of the invention, live multipotent stem and progenitor cells which have integrated into their genome DNA comprising a regulatory sequence of a mammalian nestin gene operably linked to a gene coding for a marker/reporter protein are contacted with a compound to be screened. In the absence of cell destruction, a decrease in the marker/reporter protein measurement (e.g., fluorescence) observed in cells exposed to the compound, compared to the measured marker/reporter protein of control cells is indicative of the compound's ability to promote (enhance, increase) differentiation of multipotent stem and progenitor cells into cells that no longer express the nestin gene.

A compound's ability to inhibit (decrease) differentiation is indicated by a prolonged measurement of the marker/reporter protein. In the embodiment in which the marker/reporter protein employed is a fluorescent protein, decreased differentiation of multipotent stem and progenitor cells in the presence of the compound is indicated by prolonged fluorescence in cells in the presence of the compound compared to the fluorescence of control cells. In other words, cells in the presence of a compound which inhibits differentiation will fluoresce for a longer period of time than control cells.

In a preferred embodiment of the invention the isolated cells are neural stem and progenitor cells and a decrease or increase in the marker/reporter protein measurement (e.g., fluorescence) observed when these cells are exposed to the compound, compared to the marker/reporter protein measurement (e.g., fluorescence) of control cells, is indicative of the compound's ability to promote or retard differentiation of neural stem and progenitor cells into neurons and glial cells.

In another embodiment, cells in developmental stages that precede the expression of the nestin gene (e.g. totipotent cells) can be used to screen compounds that promote their differentiation into cells that express the nestin gene, e.g. multipotent stem and progenitor cells. For example, totipotent cell differentiation after exposure to a compound can be assessed for enhanced fluorescence or enhanced presence of another marker/reporter protein as compared to control totipotent cells which have not been contacted or exposed to the compound. In a preferred embodiment, the multipotent stem and progenitor cells include neural stem and progenitor cells.

Totipotent cells can be isolated from the non-human transgenic mammal, progeny thereof or from the non-human transgenic mammalian embryo of the invention. Examples of techniques employed in isolating totipotent cells include: culturing ES cells, dissociating Blastocysts, FACS sorting based on totipotent specific promoter driving the expression of a fluorochrome, totipotent specific cell surface marker selectionby antibody, FACS, magnetic bead, affinity columns or antibody affixed to petri dish.

As known in the art, the control totipotent cells are matched to the totipotent cells contacted with the compound in every other respect except the presence of the compound being assessed. Examples of compounds that can be screened for promoting the differentiation of totipotent cells include those described above. In a preferred embodiment, the compound to be assessed is selected from the group consisting of a growth factor, a neurotrophin and a therapeutic agent.

Compounds can also be assessed for their toxicity to multipotent stem and progenitor cells, for example to neural stem and progenitor cells. Live cells are contacted with the compound to be assessed and the marker/reporter protein measurement (e.g., fluorescence) observed from these cells is compared to the fluorescence of control cells. By itself, a decrease in the measurement (e.g., fluorescence) of cells in the presence of the compound can be indicative of both cell destruction by the compound as well as cell differentiation to cell types which no longer express nestin. In a preferred embodiment of the invention, cell destruction is measured and by an independent technique, such as known to one skilled in the art. For example, if fluorescence is employed as the marker/reporter protein measurement, a non-fluorescent technique is used to measure cell destruction. A decrease in the marker/reporter protein measurement (e.g., fluorescence), coupled with a reduction in the number of live cells in the cells contacted with a compound being assessed for toxicity, when compared to the fluorescence and the number of live control cells (not contacted with the compound) is indicative of the compounds toxicity to the multipotent stem and progenitor cells or, in a preferred embodiment, to the neural stem and progenitor cells.

The present invention is further illustrated by the following examples which are not intended to be limiting. All references cited herein are incorporated by reference in their entirety.

### EXEMPLIFICATION

### EXAMPLE 1

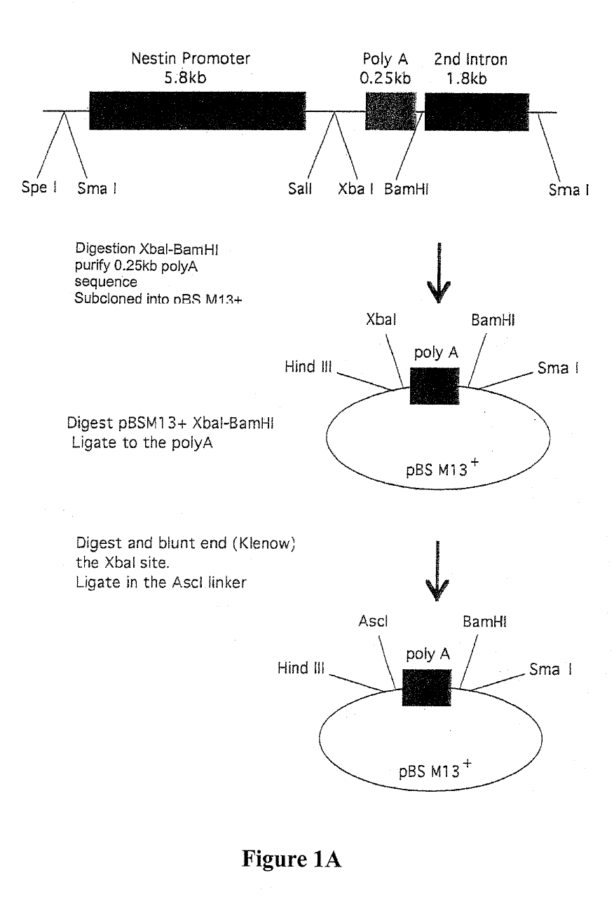

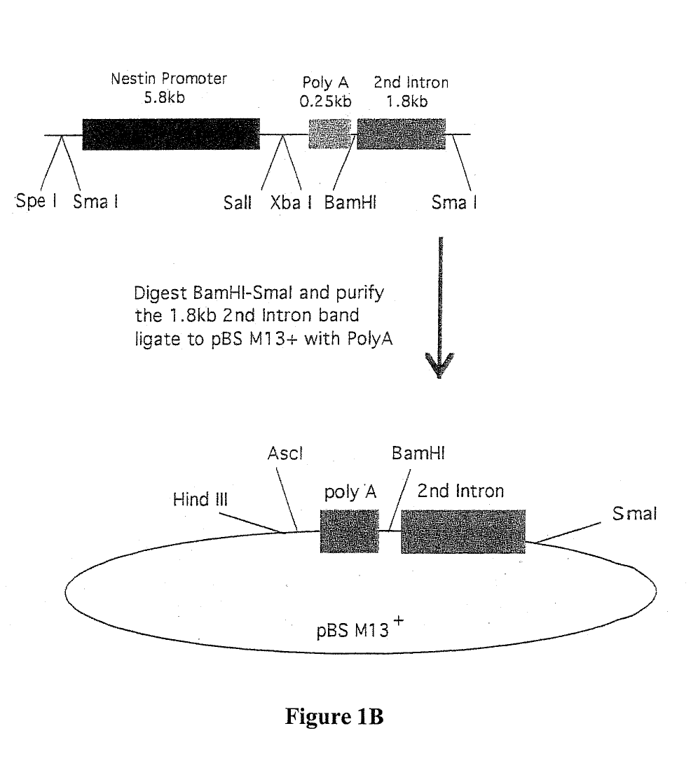

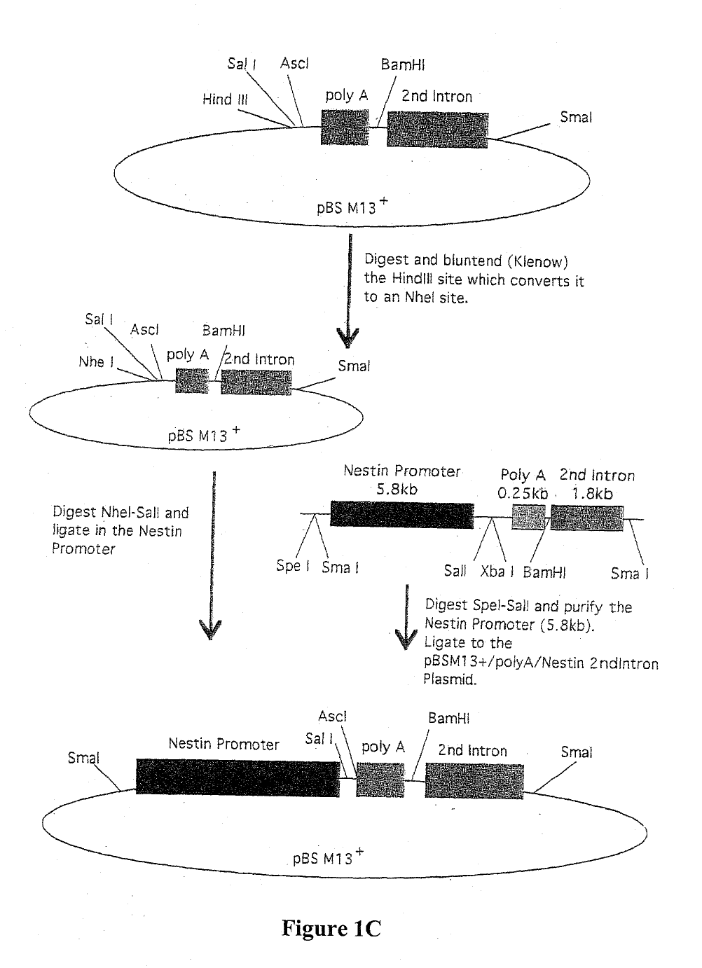

Subcloning of the nestin promoter, the poly adenylation sequence from SV40 and the second intron from the nestin gene is shown in FIGS. 1A, 1B and 1C and was performed as described below.

The SV40 splicing/polyadenylation region was removed from a plasmid bearing the nestin promoter (Zimmerman, L., et al., Neuron, 12: 11-24 (1994)), poly A, and 2nd intron of the nestin gene, by cleavage with the XbaI and BamHI restriction enzymes, revealing a 250 nucleotide base pair band, and was ligated into the pBSM13+ vector (commercially available from Stratagene) which had also been cleaved by XbaI and BamHI. The XbaI site of this polyA-pBSM13+ plasmid was then blunt ended by treatment with Klenow DNA polymerase and a linker for AscI (the sequence of which is pAGGCGCGCCT) (SEQ ID. NO.: 1) was cloned into this site, reestablishing the XbaI sites on either side of the now present AscI restriction site. The second intron (1.8 kb nucleotides) was digested by cutting the rat Nestin promoter/polyA/2^{nd} intron plasmid with the restriction enzymes BamHI and SmaI, and was then ligated 3′ to the poly-A-pBSM13+ plasmid which had also been cleaved using the BamHI and SmaI restriction enzymes. In order to clone the promoter sequence into the polyA/2^{nd} intron/pBSM13+ plasmid, the HindIII site in the polyA/2^{nd} intron/pBSM13+ plasmid was blunt ended and re-ligated, thus creating an NheI site. The nestin promoter (5.8 kb nucleotides) was then digested from the rat nestin promoter/ployA/2^{nd} intron plasmid by digesting with SpeI-SaII restriction enzymes, and was ligated to the polyA/2^{nd} intron/pBSM13+ plasmid which had been digested with the NheI-SaII restriction enzymes, placing the nestin promoter 5′ to the poly-adenylation site. The SpeI restriction site is compatible with the NheI site. In this manner, a plasmid bearing the promoter, and 2^{nd} intron elements of the rat nestin gene with an SV40 polyadenylation sequence placed between the two was created.

The pEGFP-N1 plasmid (Clontech) was used as the source for GFP The plasmid codes for a mutated version of GFP which has enhanced fluorescence. In order to subclone this gene into the rat nestin promoter/polyA/2^{nd} intron/pBSM13+ plasmid, the NotI restriction site, which is 3′ to the GFP translational stop codon, was digested with the NotI restriction enzyme, blunt ended by Klenow DNA polymerase, and an AscI linker (as above) was ligated to the site. This created an AscI restriction site in place of the NotI site. The XmaI restriction site, which can be found in the polylinker which is 5′ of the GFP gene, was blunt ended (as above) and religated in order to destroy the SmaI site. The EGFP was then digested with the restriction enzymes SaII and AscI creating a 780 bp DNA fragment, and was ligated to the nestin promoter/EGFP/SV40 polyA/2^{nd} Intron/pBSM13+ plasmid (which had been digested with SaII and AscI) 3′ to the Nestin promoter and 5″ to the polyA site.

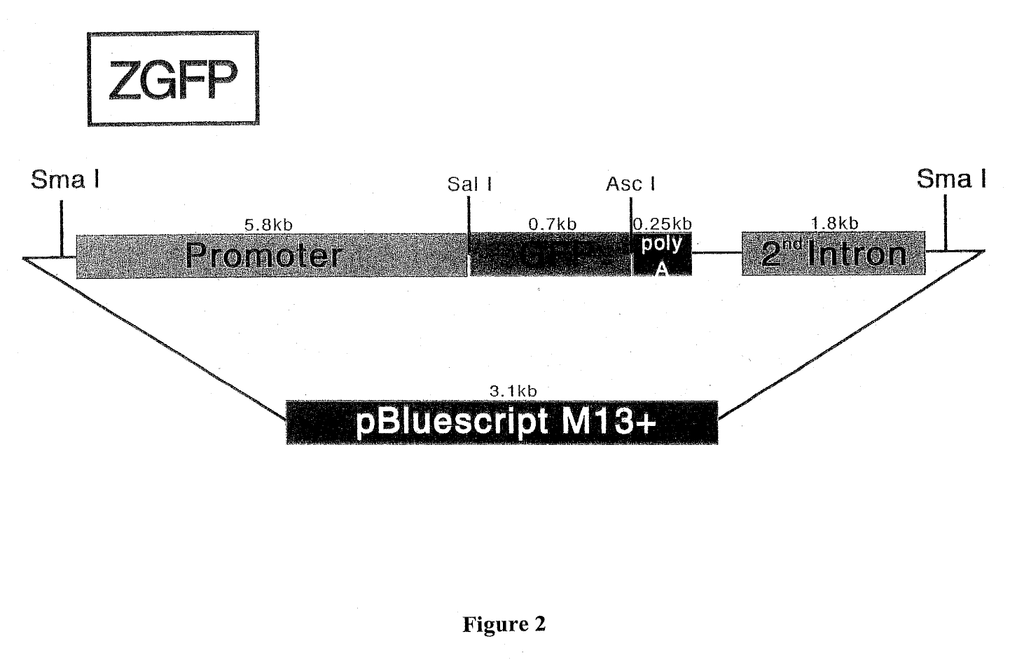

Approximately 10 μg of the plasmid was digested with the restriction enzyme SmaI. The plasmid containing the Nestin promoter/EGFP/SV40 polyA/2^{nd} Intron/pBSM13+, also referred to herein as “zGFP”, was prepared by cesium chloride centrifugation. The complete plasmid of the nestin promoter -EGF-N1-SV40PolyA-Nestin 2^{nd} intron-pBSM13+ (ZGFP) is shown in FIG. 2. To linearize, Sma I is cut to obtain a 8.55 kb fragment of the promoter, GFP and second intron; the 3.1 kb band is pBLUESCRIPT backbone. The DNA fragment containing the Nestin promoter-EGFP-polyA^{/2ND} intron was purified by agarose gel.

### EXAMPLE 2

The specific fragment, obtained as described in Example 1 above, was introduced into pronuclei of 500 oocytes of the C57BL/6× BALB/cBy hybrid strain. The injected oocytes were then transferred to 12 pseudo pregnant females. A total of 86 Fₒ pups were created by this procedure.

For the transgene detection, PCR analysis of DNA isolated from the tails was performed. The sequences of the primers used for PCR were CCTCTACAAATGTGTGATGGC (corresponding to the SV40 polyadenylation region) (SEQ ID. NO.: 2), and GCGCACCATCTTCTTCAAGGACG (corresponding to the EGFP sequence) (SEQ ID. NO.: 3). PCR was performed in 30 μl containing 10% DMS, 2.5 mM MgCl₂, 1× PCR buffer, 0.2 nM of each dNTP, 0.4 μM of each primer and 1 u amplitaq (Boeringer Mannheim). 44 cycles of PCR with an annealing temperature of 55° (30 s) and an extension temperature of 65° (1 min) were used. Under these conditions, the expected fragment of 470 bp was detected in eight out of the 86 F₀ mice. Of these eight transgenic mice, three were male and five were female.

### EXAMPLE 3

In order to evaluate whether the expression of EGFP in these positive transgenic mice, is spatially and temporally controlled by the Nestin promoter and 2^{nd} intron, the three transgene positive males were subject to matings with 3-6 week old C57BL/6 females. The establishment of a copulative plug was determined to be E0.5. Embryos were processed by timing their age past the appearance of a copulative plug in the mother. Upon proper maturation, mothers were sacrificed by CO₂ followed by cervical dislocation. The embryos were removed and placed in 0° C. PBS to wash, followed by 4% paraformaldehyde at 4° C. overnight. After paraformaldehyde treatment, the embryos were either used for whole mount analysis or placed in 30% sucrose until 24 hours after the embryo had already completely submerged (typically two days). Cryostat sections were performed by embedding the embryos in O.C.T. (optimal cutting temperature) compound (obtained from VWR) followed by sectioning using a Leica Jung Frigocut 2800 E cryostat with a box temperature of −20° C. and an objective temperature of −17° C. Sections were 40-60 μm thick and were adhered to gelatin subbed slides. E10.5, E13.5, and E16.5 embryos were analyzed for fluorescence. Whole mount images were observed using a Leica MPS30 dissecting microscope, under 0.8× objective, with an attached Mercury lamp and GFP filter. Sectioned tissue was analyzed using a Zeiss axiophot microscope with FITC filters and an attached CCD spot camera (Diagnostic Instruments); since tissues appeared much larger than the field of view of the microscope, section were taken under a 10× magnification lens and recomposed, as mosaics, in Adobe Photoshop.

Adult brains were processed first by perfusion of 4% paraformaldehyde of the mouse. Brains were then dissected from the cranium and were further treated with 4% paraformaldehyde for 4 hours at 4° C. After fixation, brains were immersed in 30% sucrose until one day post submerging (typically three days). Sagittal cryostat sections were performed using methods as stated above, however, with a box temperature of −30° C. and an objective temperature of −27° C.

### EXAMPLE 4

Nestin expression was determined to mark neural epithelial cells from as early as the onset of neural plate formation at embryonic day 7.5. In order to evaluate whether the expression of EGFP in these positive transgenic mice was spatially and temporally controlled by the Nestin promoter and 2^{nd} intron, the three transgenic positive males were subjected to matings with 3-6 week old C57BL/6 females. The establishment of a copulative plug was determined to be E 0.5. Embryos were processed by timing their age past the appearance of a copulative plug in the mother. Upon proper maturation, mothers were sacrificed by CO₂ followed by cervical dislocation. The embryos were removed and placed in 0° C. PBS to wash followed by direct whole mount microscopy. Mouse embryos were prepared in this way for embryonic days (E) 9.5, 12.5, 14.5, 15.5, 16.5, and 18.5. Whole mount images were observed using a Leica MPS30 dissecting microscope, under 0.8× objective, with an attached Mercury lamp and GFP filter. The expression of the GFP was observed throughout the entire region of central nervous system at these stages, including the retina, lens, and spinal cord. The expression began to diminish after E 12.5, which correlated well with the developmental scheme of central nervous system development. Neural differentiation initiated at this period, resulting in a noticeable decrease in the stem cell form and an increase of the differentiated form. The scheme was more clearly represented by analysis of coronal sections of the embryonic brain, which were prepared as above. However, the mice were not left in PBS but rather fixed by 4% paraformaldehyde at 4° C. overnight. After paraformaldehyde treatment the embryos were placed in 30% sucrose until 24 hours after the embryo completely submerged (typically two days). Cryostat section were performed by embedding the embryos in O.C.T. (optimal cutting temperature) compound (Sigma) followed by sectioning using a Leica Jung Frigocut 2800 E cryostat with a box temperature of −20° C. and an objective temperature of −17° C. Sections were 30 μm thick and were adhered to gelatin subbed slides. E12.5, E14.5, E15.5 and E18.5 embryos were analyzed for fluorescence. Sectioned tissue was analyzed using a Zeiss axiophot microscope with FITC filters and an attached CCD spot camera (Diagnostic Instruments); since tissues appeared much larger than the field of view of the microscope, section were taken under a 10× magnification lens and recomposed, as mosaics, in Adobe Photoshop.

Similar images were prepared for the adult mouse. Adult brains were processed first by perfusion with 4% paraformaldehyde of the mouse. Brains were then dissected from the cranium and were further treated with a 4% paraformaldehyde for 4 hours at 4° C. After fixation, brains were immersed in 30% sucrose until one-day post submerging (typically three days). Sagittal cryostat sections were performed using methods as stated above, however with a box temperature of −30° C. and an objective temperature of −27° C. Adult mice (6 weeks) showed expression of GFP in the regions of the brain previously reported as the sites of neoneurogenesis as previously identified by BrdU incorporation, such as the dentate gyrus (Kempermann et al., Proc. Natl. Acad. Sci., Vol. 94(19):10409-10414 (1997), the subventricular zone (Morshead, et al., Neuron, Vol. 13(5):1071-1082 (1994), the olfactory bulb, and the rostral migratory stream (Subonen, et al., Nature, Vol. 383(6601):624-627 (1996).

### EXAMPLE 5

In order to determine whether the gene for EGFP was both spatially and temporally expressed in neuronal stem cells, immunohistochemistry was employed.

The central nervous system of a mouse is derived from a strip of ectoderm, rostral to the primitive streak. This tissue becomes the neural plate, appearing at 7.5 days of embryogenesis. The neural plate undergoes rapid cellular growth and by the ninth and tenth day of embryogenesis, the cells on opposite sides of the neural plate fuse to form the neural tube. The cavity of the neural tube eventually becomes the ventricle of the maturing and adult brain and the central canal of the spinal cord. It is from this interface of cavity to tissue that the major divisions and development of the brain occurs. As the cells migrate tangentially from the surface of the ventricle, their fate becomes more specific with more symmetric cellular divisions. Several antigens are known which can determine the stage of development of the major cell types in the brain (typically neural stem, neuron, astroglia, and hematocytes).

In these experiments, the several different phenotypes of the brain were identified and compared in terms of amount and place by employing an immunohistological approach. In order to determine whether these fluorescently tagged cells were indeed representative of cell type, namely nestin-positive (nestin⁺) cells and neural stem cells, the GFP cells were compared versus a marker for differentiated neurons (βIII tubulin) and a marker for differentiated astrocytes (GFAP). These comparisons allowed the analysis of whether the Nestin⁺/GFP⁺ cells of the brain in both embryogenic and adult stages, colocalized. The results indicated a very apparent shift in the developmental stages, where in early embryogenesis, the cells immediately surrounding the ventricles were GFP⁺, while more distal to the ventricle this expression pattern had a very obvious down regulation of the GFP⁺ expression and an increase in β III tubulin immunohistochemistry. The nestin promoter sufficiently allowed GFP expression to those regions of the brain surrounding the ventricle.

In the adult, the expression pattern of the Nestin promoter regulation of GFP largely decreased. The colocalization pattern with those aforementioned markers, βIII-tubulin and GFAP was found intermixed, however largely not colocalizing. In the posterior region of the lateral ventricle there was a high degree of regional colocalization with the astrocyte marker GFAP however very little cellular colocalization. In the anterior regions of the lateral ventricle, where the cells began to radiate from the ventricle into the rostral migratory stream, there was a higher degree of regional colocalization with the neuronal marker βIII-tubulin than GFAP.

As the cells migrated along the rostral migratory stream, again, there was a high degree of expression of Nestin-GFP; however, there was little to no colocalization of GFP with either GFAP of Tubulin markers.

These results support the belief that a large number of new cells being produced by the lateral ventricle become neurons as well as the belief that certain forms of astrocytes serve as symmetrically dividing progenitor cells. Also, these results indicate that the nestin-GFP transgene is not colocalizing with markers of a differentiated phenotype.

### EXAMPLE 6

Transgenic positive males were mated with C57B6 female mice. The appearance of a copulative plug labeled the fertilized embryos of the female to be in embryonic day 0.5 of development. At 13.5 days of embryogenesis, the mother was sacrificed and the embryos were removed. Crown to rump measurements were taken in order to show that no discrepancy existed between the development of the transgenic positive and negative littermates. No size phenotype could be found among the pups. The embryos were washed in 4° C. PBS and a hand held UV light was used to determine which mice were transgenic and which were not. At this point, the entire central nervous system is expressing high levels of GFP and transgenics can easily be determined through this UV method. The brain tissue was removed from the fetus and placed in Hank's Buffered Salt Solution (HBSS) (Gibco) 4° C. to a total of 5 ml. This solution was then mixed 1:1 with a 2× trypsin solution, containing 0.25% trypsin (Gibco), 1 mM EDTA (Gibco), and 1 mg/ml collagenase (Gibco) all in HBSS. This solution was incubated for 15 minutes at 37° C. with agitation every three minutes. In order to quench enzymatic digestion activity, 0.1 mg/ml ovomucoid (Sigma) was added. The tissue was then triturated using a 19, and then a 21 gauge syringe. The cells were then pelleted 10 minutes 500 rpm in a Beckman table top centrifuge at 4° C. The cells were then resuspended in ice cold PBS at 1×10⁶ cells/ml. In this way, two samples of primary cells were prepared, one taken from the brain tissue of positive nestin/GFP mice and one taken from the brain tissue of negative nestin/GFP mice. Both samples are derived from the embryos of the same mother (littermates).

Fluorescence activated cell sorting (FACS) was performed by the Coulter Elite ESP FACS. Cells were kept on ice in PBS except for the period while sorting and collecting, for the duration of these experiments. The cells were placed through a 70 micrometer nylon mesh to remove clumps of cells and then run through the machine. The filter used for GFP detection was the photomultiplier tube 2 (PMT2) which has a wavelength emission between 520 and 530 nm.

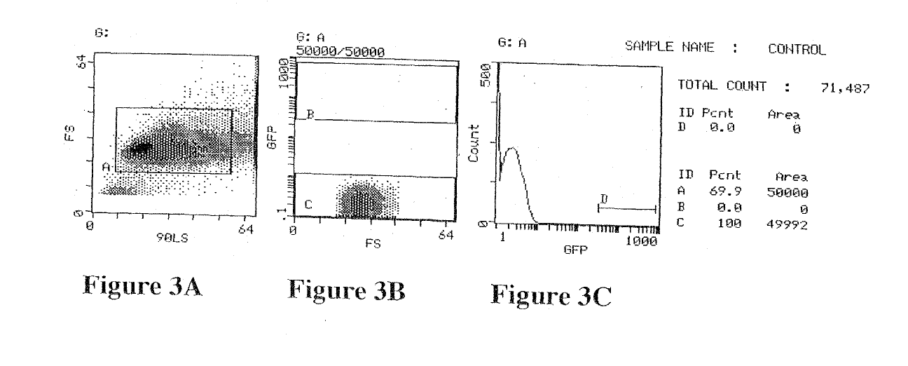

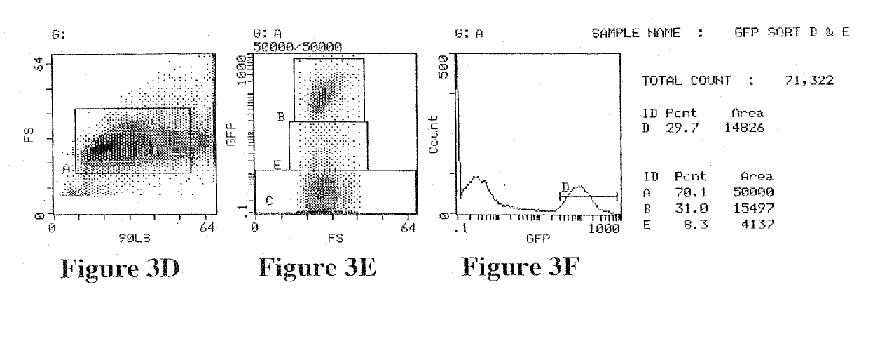

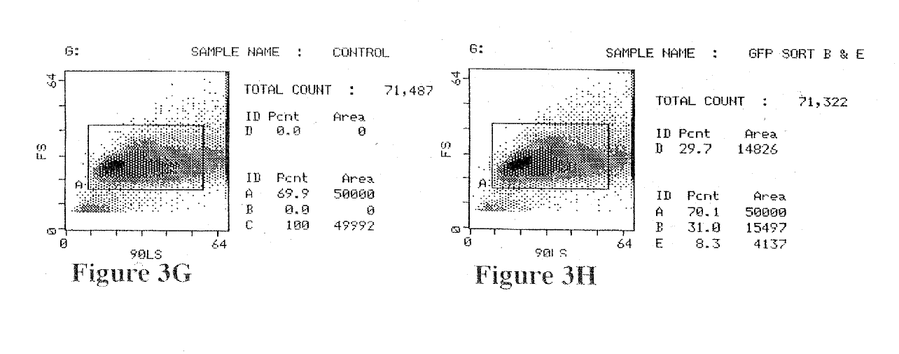

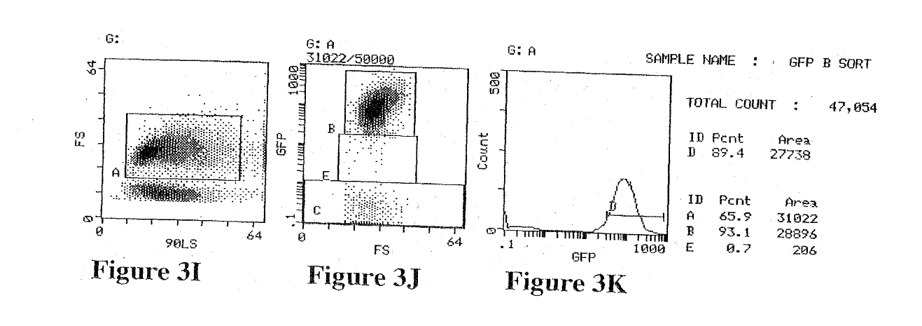

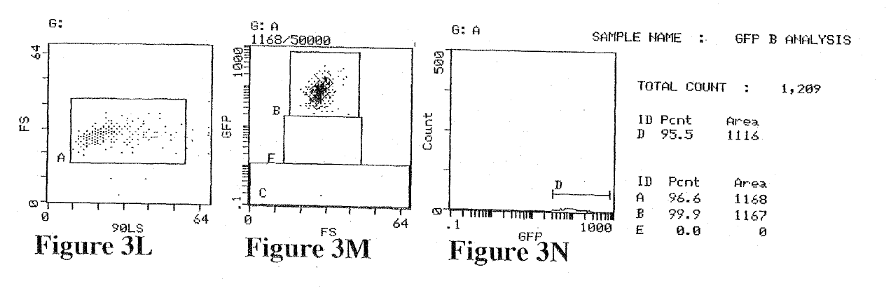

FACS results are shown in FIGS. 3A-3N. The cells derived from the Nestin-GFP negative embryos were first analyzed by the FACS machine in order to establish the background levels of fluorescence, as well as to determine the size and shape of the cells of this neuronal population. The results are shown in FIGS. 3A-3C which display what non-transgenic embryonic brain cells (control cells) appeared when run through the FACS machine.

FIG. 3A displays the size (forward scatter or FS) and shape (side scatter or LS) of the cells in the population. Box A shows where the bulk of healthy, non-clumped cells was found, as determined by prior history of FACS work. Each dot signified a data point (an actual cell). Typically, points at the bottom of the FS axis represented cellular debris, while points to the extreme right on the LS axis represented clumps of cells. The box “A” enclosing 69.9% of the population of cells represented the pool of cells that were analyzed in the next data panels. Cells lying outside of this box which may have been clumps, dead or cellular debris were not included.

Data points shown in FIG. 3B were those gated by Box “A” in the left hand panel. “Gated” means that only those cells that lie within box “A” of FIG. 3A were analyzed in FIG. 3B. FIG. 3B shows the relative degree of GFP fluorescence (vertical axis) versus the FS or cell size (horisontal axis) of cells from gate A of FIG. 3A. By placing box “C” around this population of cells, background fluorescence was marked. Gate C was then used as a marker for background fluorescence for the rest of the experiments. Any point registering above this C gate represented a cell that had a higher fluorescence intensity than background, and therefore, was GFP positive, since GFP was the only source of fluorescence in the subsequent experiments. (There was no fluorescence of non-transgenic mouse cells outside of the box labeled “C.”) FIG. 3C shows the cell number on the vertical axis in comparison to the fluorescence intensity of GFP in a log scale on the horizontal axis and indicate that the population of non-transgenic cells derived from the brain showed no GFP intensity.

FIGS. 3D-3E show the FACS analysis of cells from transgenic littermates. Typically, the pregnant mouse had about nine (9) embryos. When these embryos were removed for these experiments, the embryos that were positive for the transgene were discerned from those that were negative by using a handheld UV lamp. The positives had a characteristic fluorescent pattern through the central nervous system. FIG. 3D is the same as that for the control after a similar number of cells have been analyzed (71,322). (This is shown side by side in FIG. 3G and FIG. 3H which illustrates that the populations of the positive and negative cells are identical in terms of forward and lateral scatter.) The Nestin GFP cells displayed an equivalent size and shape as those of their non-transgenic littermates. 70.1% of all cells were found within gate A in the transgenic tissue as compared to 69.9% of cells found in gate A for non-transgenic cells.

FIG. 3E shows that the Nestin-GFP positive cells displayed two obvious populations, one population within gate C, thus, a population of non-GFP expressing cells (similar to the background population) and another, within gate B, displaying 100× greater fluorescence than background. Of the 71,322 cells analyzed, 41.1% had a higher fluorescent intensity than the control cells. The second population, as denoted by box “B”, included 31.0% of the cells. The cells of Box “B” were sorted (or, rather, isolated) by the FACS machine.

FIG. 3F displays the two peaks and shows a region of high GFP fluorescence (marked as gate D). This data demonstrated that the Nestin-2nd intron transcriptional unit was active in 39.3% of cells derived from the embryonic day 13.5 mouse brain in this experiment. Also, of this 39.3%, 31% of the cells were 100× more fluorescent than background cells.

The next experiment used the FACS machine to further purify the population of high GFP fluorescence. The cells from box B in the experiment described above were sorted again. The cells were sorted by both gates A and B to ensure a population of healthy, highly fluorescent single cells. The results are shown in FIGS. 3I-3K. As a result of the sort, cells of Gate B from the above experiment (31% of the total population) now contained about 93.1% of the total population which exhibited high GFP fluorescence.

To determine the purity of cells isolated by two rounds of cell sorting, cells in gate B (FIG. 3J) were pelleted and resuspended in a smaller volume of PBS. This population was analyzed again by FACS. sorted again. The results are shown in FIGS. 3L-3N. 1,289 cells were analyzed and, of this group, 99.9% of the cells had high GFP fluorescence. This demonstrated that sorted cells (Gate B, FIG. 3J) represented a highly purified population which expressed GFP transgene at high level.

### EXAMPLE 7

Experiments based on neurosphere colonies formation were also conducted in order to determine whether a Nestin GFP cell is a stem cell or a progenitor cell. The neurosphere colony formation is a common assay originally devised by Reynolds and Weiss, Science, Vol. 255:1701-1710 (1992), the entire contents of which are incorporated herein by reference, and is used to determine whether a cell is indeed a neural stem cell. By growing primary cell cultures from enzymatically dissociated brain tissue, one is able to identify cells that would create large spherical colonies in culture when placed in a serum free medium with EGF. These spherical masses could then be plated on laminin coated coverslips with fetal bovine serum in the medium where they initiated adherence to the surface with subsequent differentiation of the cells into three different neural cell phenotypes (neurons, astrocytes, and oligodendrocytes) within five to seven days. These differentiated cells no longer undergo mitosis, and no longer express the stem cell marker Nestin.

The procedure employed in these experiments was as follows. Cells were harvested from the adult transgenic C57BL/6 mouse strain typically using mouse line #33. The mouse was injected (intraperitoneal) with 400 μl of 15% Chloryl Hydrate as a sedative. Upon loss of reflex, the mouse was perfused with ice cold 10 ml of Hanks buffered salt solution (without calcium or magnesium) to remove the blood. The brain was then dissected out of the cranium and placed in HBSS^{-/-}. The region of the ventricle was prepared by two coronal dissection removing the olfactory bulb and the cerebellum. The block of tissue was then diced using a no. 10 scalpel. The tissue was then placed in DMEM/F12 media with 100 u/ml penicillin G/sodium and 100 μg/ml streptomycin sulfate (Gibco) for five minutes on ice. The tissue was allowed to settle and all the supernatant was removed. Six milliliters of trypsin 0.025% (Gibco) was then added in the presence of versene and allowed to enzymatically digest the tissue for ten minutes. The cells were then triterated by pipette until they were single cells, as determined by a hemostat. The cells were pelleted three times by adding DMEM/F12 and centrifuging to remove the trypsin and were then resuspended in M21 serum free media and supplemented with 20 ng/ml EGF. The cells were then plated at a density of 20,000 cells per milliliter of media, or lower for clonal density. The media was then supplemented with 1000 ng EGF every three days.

The neurospheres appeared after five days and had a 50-80 μm diameter by two weeks. It was routinely possible to achieve high numbers of neurospheres in this manner, all of which were highly GFP positive. Upon plating these fluorescing spheres on laminin in the presence of FBS, differentiation of various other cell types such as nonfluorescing neurons and astrocytes were obtained. The nestin-GFP derived cells of the lateral ventricle were able to form neurospheres as well as to be induced to give multiple phenotypes upon being induced to differentiate, indicating that the Nestin-GFP cells were neural stem cells.

### EXAMPLE 8

Neuronal transplant experiments also were performed. In these experiments nestin-GFP⁺ cells purified or isolated from a transgenic mouse were inserted into the brain of a recipient animal (in these studies a Sprague Dawley rat was used). Experiments were performed to evaluate whether nestin-GFP cells could be transplanted to the recipient rat brain and, whether these cells could not only survive but also incorporate themselves into the normal developmental scheme of the region to which they were delivered.

The embryonic mouse was prepared by CO2 asphyxiation of the mother followed by removal of the embryos immediately into ice cold HBSS. Transgenic positive embryos were determined by a hand held UV lamp, and the entire head was removed. The tissue was then placed in DMEM/F12 media with 100 u/ml penicillin G/sodium and 100 μ/ml streptomycin sulfate (Gibco) for five minutes on ice. The tissue was allowed to settle and all the supernatant was removed. Six milliliters of trypsin 0.025% (Gibco) was then added in the presence of versene and allowed to enzymatically digest the tissue for ten minutes. The cells were then triterated by pipette until they were single cells, as determined by a hemostat. The cells were then be pelleted three times by adding DMEM/F12 and centrifuging to remove the trypsin. An embryonic day 14 mouse (which was 60% GFP⁺ in the CNS at this period) was disrupted. The cells were diluted to 20,000 cells in 10 μl and kept on ice in HBSS^{-/-} until the recipient rat was ready for cellular transfer. The adult rat was prepared by anesthetizing the rat using a mixture of ketamine and xylazine at 100 mg/kg and 10 mg/k, respectively, diluted in saline and injected IP. The degree of sedation was checked by paw reflex through pinching. When sedate, the animal's head was shaved using an electric razor and positioned in a stereotactic frame. The rat's mouth was then opened and the incisors were inserted across the tooth bar. The earbars were placed on either side into the auditory canal and locked into place. Next, the nose clamp was tightened over the animal's snout and the skin was cleaned with betadine. Using a no. 10 scalpel, a midline incision was made 1.5 cm in the scalp and using forceps and fine scissors, and starting at the midline and move laterally, the pericranium was removed to expose the skull. Using the intersection of the coronal and sagittal sutures, the bregma was identified for use as a stereotactic reference point. The 20,000 cells were then loaded into a Hamilton which was then attached to the holder in the stereotactic frame. The tip of the needle was moved to the bregma to record the AP and ML coordinates (using Paxinos and Watson 1982 for coordinates) and then moved to the stereotactic coordinates for the lateral ventricle. At this location, a hole was drilled using a number 1 carbide bur affixed to a dental drill at the marked spot. The needle was next lowered to the very top of the dura to record its vertical coordinate and lowered ventrally at approximately 1 mm/min. The cells were injected at approximately 1 μl/min; the needle was left for 4 min and then removed at a rate of 1 mm/min. Next, the skin was irrigated at the point of removal with saline and closed with a non-absorbable number 2.0 ethilon sutures using a reverse cutting needle and covered in antiseptic gel. In this manner cells could be injected into the ventricle accurately; the method allowed also directed injections by adjusting the AP and ML coordinates.

It was found after a one-week survival period that the cells were able to survive and incorporate into the brain.

While this invention has been particularly shown and described with references to preferred embodiments thereof, it will be understood by those skilled in the art that various changes in form and details may be made therein without departing from the scope of the invention encompassed by the appended claims.

What is claimed is:

1. A non-human transgenic mammal, progeny or embryo thereof which has integrated into its genome DNA comprising a regulatory sequence of a mammalian nestin gene operably linked to a gene coding for a fluorescent protein wherein the gene coding for the fluorescent protein is expressed in multipotent stem and progenitor cells of the non-human transgenic mammal, progeny or embryo thereof.

2. The non-human transgenic mammal, progeny or embryo thereof of claim 1 wherein the gene coding for the fluorescent protein is selectively expressed in multipotent stem and progenitor cells of the non-human transgenic mammal or progeny thereof.

3. The non-human transgenic mammal, progeny or embryo thereof of claim 1 wherein the gene coding for the fluorescent protein is expressed in neural stem and progenitor cells of the non-human transgenic mammal or progeny thereof.

4. The non-human transgenic mammal, progeny or embryo thereof of claim 1 wherein the mammal is mouse.

5. The non-human transgenic mammal, progeny or embryo thereof of claim 1 wherein the regulatory sequence of the mammalian nestin gene is obtained from rat nestin gene.

6. The non-human transgenic mammal, progeny or embryo thereof of claim 1 wherein the regulatory sequence includes a second intron sequence of the mammalian nestin gene.

7. The non-human transgenic mammal, progeny or embryo thereof of claim 1 wherein the regulatory sequence includes a promoter.

8. The non-human transgenic mammal, progeny or embryo thereof of claim 7 wherein both the promoter and the regulatory sequence are obtained from the same mammalian nestin gene.

9. A method of producing a non-human transgenic mammal which expresses a fluorescent protein in multipotent stem and progenitor cells, comprising:
(a) introducing into a fertilized egg of a non-human mammal, DNA comprising a regulatory sequence of a mammalian nestin gene operably linked to a gene coding for a fluorescent protein that is expressed in multipotent stem and progenitor cells of the non-human mammal;
(b) introducing the fertilized egg of (a) into a non-human mammal of the same species;
(c) allowing the non-human mammal to produce progeny which are non-human transgenic mammals; and
(d) selecting non-human mammal progeny of (c) whose multipotent stem and progenitor cells express the fluorescent gene.

10. The method of claim 9 wherein the gene coding for a fluorescent protein is selectively expressed in multipotent stem and progenitor cells.

11. The method of claim 9 wherein the gene coding for a fluorescent protein is expressed in neural stem and progenitor cells.

12. The method of claim 9 wherein the non-human transgenic mammal is mouse.

13. The method of claim 9 wherein the the regulatory sequence of the mammalian nestin gene is obtained from rat nestin gene.

14. The method of claim 9 wherein the regulatory sequence comprises a second intron sequence of the mammalian nestin gene.

15. The method of claim 14 wherein the regulatory sequence further includes a promoter.

16. The method of claim 15 wherein both the promoter and the regulatory sequence are obtained from the same mammalian nestin gene.

17. A non-human transgenic mammal produced by the method of claim 9.

18. An expression construct comprising a promoter sequence, a gene coding for green fluorescent protein and a regulatory sequence present in the second intron of a mammalian nestin gene.

19. A cell comprising an expression construct which includes a promoter sequence, a gene coding for green fluorescent protein and a regulatory sequence present in the second intron of a mammalian nestin gene.

20. A method for measuring a multipotent stem and progenitor cell population in an animal organ or region thereof, comprising:
measuring cells which fluoresce from the organ or region thereof of a non-human transgenic mammal which has integrated into its genome DNA comprising:
a regulatory sequence operably linked to a gene coding for a fluorescent protein, wherein the gene coding for the fluorescent protein is expressed in multipotent stem and progenitor cells of the non-human transgenic mammal,
wherein the cells which fluoresce are multipotent stem and progenitor cells.

21. The method of claim 20 wherein the gene coding for a fluorescent protein is selectively expressed in multipotent stem and progenitor cells.

22. The method of claim 20 wherein the gene coding for a fluorescent protein is expressed in neural stem and progenitor cells.

23. The non-human transgenic mammal, progeny or embryo thereof of claim 20 wherein the regulatory sequence includes a second intron sequence of the mammalian nestin gene.

24. The non-human transgenic mammal, progeny or embryo thereof of claim 20 wherein the regulatory sequence further includes a promoter.

25. The non-human transgenic mammal, progeny or embryo thereof of claim 24 wherein both the promoter and the regulatory sequence are obtained from the same mammalian nestin gene.

26. A method of obtaining primary, noncultured, multipotent stem and progenitor cells comprising isolating cells which express a marker/reporter protein from a non-human transgenic mammal, progeny or embryo thereof which has integrated into its genome DNA comprising a regulatory sequence of a mammalian nestin gene operably linked to a gene coding for the marker/reporter protein wherein the gene coding for the marker/reporter protein is expressed in multipotent stem and progenitor cells of the non-human transgenic mammal, progeny or embryo thereof.

27. Cells obtained by the method of claim 26.

28. A method of obtaining primary, noncultured, multipotent stem and progenitor cells comprising isolating fluorescent cells from a non-human transgenic mammal, progeny or embryo thereof which has integrated into its genome DNA comprising a regulatory sequence of a mammalian nestin gene operably linked to a gene coding for a fluorescent protein wherein the gene coding for the fluorescent protein is expressed in multipotent stem and progenitor cells of the non-human transgenic mammal, progeny or embryo thereof.

29. The method of claim 28 wherein the gene coding for the fluorescent protein is selectively expressed in multipotent stem and progenitor cells of the non-human transgenic mammal, progeny or embryo thereof.

30. The method of claim 28 wherein the gene coding for the fluorescent protein is expressed in neural stem and progenitor cells of the non-human transgenic mammal, progeny or embryo thereof.

31. The method of claim 28 wherein the regulatory sequence comprises a second intron sequence of the mammalian nestin gene.

32. The method of claim 28 wherein the regulatory sequence further includes a promoter.

33. he method of claim 32 wherein both the promoter and the regulatory sequence are obtained from the same mammalian nestin gene.

34. The method of claim 28 further comprising identifying and/or isolating genes expressed in said isolated fluorescent cells.

35. The method of claim 28 further comprising identifying and/or isolating proteins expressed in said isolated fluorescent cells.

36. The method of claim 28 further comprising identifying and/or isolating cell-specific surface antigens expressed on said isolated fluorescent cells.

37. The method of claim 28 further comprising transplanting said isolated fluorescent cells into a live animal or a viable embryo.

38. The method of claim 28 wherein fluorescent cells are isolated by fluorescent activated cell sorting.

39. Cells obtained by the method of claim 28.

40. A method for assessing a compound's ability to promote multipotent stem and progenitor cell differentiation, comprising:
(a) contacting live multipotent stem and progenitor cells, which have integrated into their genome DNA comprising a regulatory sequence of a mammalian nestin gene operably linked to a gene coding for a marker/reporter protein wherein the gene coding for the marker/reporter protein is expressed in multipotent stem and progenitor cells, with a compound to be assessed;
(b) determining a marker/reporter protein measurement of the live cells of a) in the presence of the compound; and
(c) comparing the marker/reporter protein measurement of b) to the marker/reporter protein measurement of live control cells;
wherein a decrease or absence of marker/reporter protein measurement of the live cells in the presence of the compound compared to the marker/reporter protein measurement of the live control cells is indicative of the compound's ability to promote multipotent stem and progenitor cell differentiation.

41. The method of claim 40 wherein the marker/reporter protein is a fluorescent protein and the marker/reporter protein measurement is fluorescence.

42. The method of claim 41 wherein the gene coding for the fluorescent protein is selectively expressed in multipotent stem and progenitor cells.

43. The method of claim 41 wherein the gene coding for the fluorescent protein is expressed in neural stem and progenitor cells.

44. The method of claim 40 wherein the compound is a therapeutic agent.

45. The method of claim 40 wherein the differentiation is to neural stem and progenitor cells.

46. A method for assessing a compound's toxicity to multipotent stem and progenitor cells, comprising:
(a) contacting live stem and progenitor cells, which have integrated into their genome DNA comprising a regulatory sequence of a mammalian nestin gene operably linked to a gene coding for a marker/reporter protein, wherein the gene coding for the marker/reporter protein is expressed in multipotent stem and progenitor cells, with a compound to be assessed;
(b) determining live cells expressing the marker/reporter protein in the presence of the compound; and
(c) comparing the live cells expressing the marker/reporter protein of b) to live, control cells expressing the marker/reporter protein;
wherein a decrease or absence of live cells expressing the marker/reporter protein in the presence of the compound compared to the live control cells expressing the marker/reporter protein is indicative of the compound's toxicity to multipotent stem and progenitor cells.

47. The method of claim 46 wherein the marker/reporter protein is a fluorescent protein and cells expressing the marker/reporter protein are fluorescent cells.

48. The method of claim 47 wherein the gene coding for a fluorescent protein is selectively expressed in multipotent stem and progenitor cells.

49. The method of claim 47 wherein the gene coding for fluorescent protein is expressed in neural stem and progenitor cells.

50. A method for assessing a compound's ability to promote differentiation of totipotent cells into multipotent stem and progenitor cells, comprising:
(a) contacting live totipotent stem and progenitor cells, which have integrated into their genome DNA comprising a regulatory sequence of a mammalian nestin gene operably linked to a gene coding for a marker/reporter protein, wherein the gene coding for the marker/reporter protein is expressed in multipotent stem and progenitor cells;
(b) determining a marker/reporter protein measurement of the live cells of a) in the presence of the compound; and
(c) comparing the marker/reporter protein measurement of b) to marker/reporter protein measurement of control cells;
wherein an increase of marker/reporter protein measurement in the presence of the compound compared to the marker/reporter protein measurement of control cells is indicative of the compound's ability to promote differentiation of totipotent cells into multipotent stem and progenitor cells.

51. The method of claim 50 wherein the marker/reporter protein is a fluorescent protein and the marker/reporter protein measurement is fluorescence.

52. The method of claim 51 wherein the gene coding for a fluorescent protein is selectively expressed in multipotent stem and progenitor cells.

53. The method of claim 51 wherein the compound is a therapeutic agent.
